(19) United States
(12) Patent Application Publication
(10) Pub. No.: US 2022/0304733 A1
(43) Pub. Date: Sep. 29, 2022

(54) RATCHETING MECHANISM FOR MEDICAL SCREWDRIVER

(71) Applicant: ECA Medical Instrument, Inc., Thousand Oaks, CA (US)

(72) Inventors: Randall Allard, Thousand Oaks, CA (US); Philippe Pare, Thousand Oaks, CA (US); Navid Mahpeykar, Thousand Oaks, CA (US)

(21) Appl. No.: 17/701,574

(22) Filed: Mar. 22, 2022

Related U.S. Application Data
(60) Provisional application No. 63/165,082, filed on Mar. 23, 2021.

(51) Int. Cl. A61B 17/88

(52) U.S. Cl. CPC A61B 17/8875; A61B 2017/00407

(57) ABSTRACT

Described herein are devices and mechanisms for effective and streamlined ratcheting of handheld medical devices, for example, medical ratcheting screwdrivers. In some embodiments, a ratcheting mechanism is included, which comprises a moveable component, which can comprise a plurality of teeth, an engagement component comprising a plurality of protrusions configured to engage with the teeth, and a toggle collar between the moveable component and the engagement component. In some embodiments the toggle collar comprises stops configured to engage the protrusions and configured to toggle the ratcheting mechanism between modes allowing transmission of torque and force in a first rotational direction, but disallowing it in a second rotational direction and a mode allowing transmission of torque and force in the second rotational direction, but disallowing it in the first rotational direction.

### RELATED APPLICATION

This application claims the benefit of U.S. Provisional Application Ser. No. 63/165,082, filed on Mar. 23, 2021. The application referred to in this paragraph is incorporated by reference as if set forth fully herein.

### BACKGROUND

### Related Field

The present disclosure is related generally to improvements to instruments for use in the medical field for surgical procedures, and specifically, to screwdrivers and other hand tool devices with integrated ratcheting mechanisms for surgical applications.

### Description of the Related Art

The use of surgical instruments, including hand screwdrivers is common in the medical industry. Surgical hand tools, such as ratcheting screwdrivers, are used for a variety of reasons in surgical settings. Ratcheting screwdrivers with gear mechanisms or the like, elongated handles and internal springs are also commonly used in the industry. Some conventional screwdrivers include an adjustment mechanism, which allows the user to ratchet in one or both directions. Most screwdrivers use a gear with gear teeth that engage two pawls, or actuators, that move in and out of the teeth. The pawls typically consist of only a few number of teeth, and over time, the teeth can roll over or wear away until the ratchet no longer functions properly. Thus, there is a need for a ratcheting screwdriver which is stronger and more durable than existing ratcheting screwdrivers.

Another issue with conventional medical ratcheting screwdrivers is that they typically employ many small moving parts, for example, metal separate components including leaf springs, coil springs, radial ratchet teeth and moveable pawls. The reliance on many separate moving components can increase the likelihood of device degradation or device failure through regular normal use.

### SUMMARY

Embodiments incorporating features of the present disclosure include medical screwdrivers comprising a ratcheting mechanism with a streamlined design to reduce the number of components, especially separate moving parts, to provide a more effective ratcheting device.

In one embodiment, a ratcheting medical screwdriver comprises a housing comprising a proximal and distal end, a handle portion at the proximal end, the handle portion configured to transmit motion and torque, an operational component at the distal end, the operational component configured to engage a medical implant, and a ratcheting mechanism between the operational component and the handle, the ratcheting mechanism configured to allow torque generated by movement from the handle in a first direction to be transmitted to the operational component and to prevent torque generated by movement from the handle in a second direction to be transmitted to the operational component.

In another embodiment, a ratcheting mechanism for use in a medical screwdriver comprises a moveable component configured to connect to a handle portion of a medical screwdriver, the moveable component comprising a plurality of teeth, an engagement component configured to connect to an operational component of the medical screwdriver, wherein the operational component is configured to engage a medical implant, the engagement component comprising protrusions configured to engage with the plurality of teeth, and a toggle collar between the moveable component and the engagement component, the toggle collar comprising a plurality of stops configured to engage with the protrusions.

In yet another embodiment, a ratcheting medical screwdriver comprises a housing comprising a proximal and distal end, a handle portion at the proximal end, the handle portion configured to transmit motion and torque, an operational component at the distal end, the operational component configured to engage a medical implant, a ratcheting mechanism between the operational component and the handle, the ratcheting mechanism comprising, a moveable component connected to the handle portion, the moveable component comprising a plurality of teeth, an engagement component connected to the operational component, the engagement component comprising protrusions configured to engage with the plurality of teeth, and a toggle collar between the moveable component and the engagement component, the toggle collar comprising a plurality of stops configured to engage with the protrusions.

These and other further features and advantages of the present disclosure would be apparent to those skilled in the art from the following detailed description, taken together with the accompanying drawings, wherein like numerals designate corresponding parts in the figures, in which:

### BRIEF DESCRIPTION OF THE DRAWINGS

FIG. 1 shows a front perspective view of an embodiment of a ratcheting medical screwdriver device incorporating features of the present disclosure;

FIG. 2 shows a sectional view of the embodiment of FIG. 1;

FIG. 3 shows a front perspective view of the ratcheting mechanism that was shown integrated into the ratcheting medical screwdriver device of FIG. 1;

FIG. 4 shows a rear perspective view of the ratcheting mechanism of FIG. 3;

FIG. 5 shows an exploded view of the ratcheting mechanism of FIG. 3;

FIG. 6 shows a rear perspective view of the ratcheting mechanism of FIG. 3, with the engagement component shown removed in order to better view internal features;

FIG. 7 shows a rear schematic view of the ratcheting mechanism of FIG. 3, with the engagement component shown removed in order to better view internal features;

FIG. 8 shows a rear operational view of the ratcheting mechanism of FIG. 3, shown toggled into a first operational configuration, with the engagement component shown removed in order to better view internal features;

FIG. 9 shows a rear operational view of the ratcheting mechanism of FIG. 3, shown toggled into a second operational configuration, with the engagement component shown removed in order to better view internal features;

FIG. 10 shows a partially-transparent front perspective view of another embodiment of a ratcheting medical screwdriver incorporating features of the present disclosure, further incorporating a torque-limiter;

FIG. 11 shows a rear view of another embodiment of a ratcheting mechanism incorporating features of the present disclosure; and

FIG. 12 shows a rear view of another embodiment of a ratcheting mechanism incorporating features of the present disclosure.

### DETAILED DESCRIPTION

In the following detailed description, numerous specific details are set forth in order to provide a more thorough understanding of embodiments incorporating features of the present disclosure. However, it will be apparent to one skilled in the art that devices and methods according to the present disclosure can be practiced without necessarily being limited to these specifically recited details. In other instances, well-known structures and devices are shown in block diagram form, rather than in detail, in order to better describe embodiments incorporating features of the present disclosure.

Devices incorporating features of the present disclosure can utilize a ratcheting mechanism, that can comprise at least three components: a moveable component, which can be connected to a handle portion, an engagement component, configured to engage the moveable component in some manner, and a toggle collar, which is configured to control the interaction between the moveable component and the engagement component.

In some embodiments the moveable component comprises a plurality of teeth. While no specific number of teeth is required in embodiments incorporating features of the present disclosure, in some embodiments and circumstances, a greater number of teeth can improve the ratcheting function of the mechanism. In some suitable embodiments, the moveable component comprises at least 40 teeth.

The engagement component can comprise a plurality of protrusions that can engage the teeth of the movement component to allow transfer of torque and rotational movement in one direction, and disallow transfer in another direction. Which directional transfer is allowed can be controlled by the toggle collar, which can comprise stops that can engage with the protrusions of the engagement component, which in turn changes how the teeth engage with the protrusions.

At least one advantage of the devices and mechanisms incorporating features of the present disclosure is that the design of the ratcheting mechanism has been streamlined to reduce the number of small moving parts needed to perform a ratcheting function. Furthermore, due to the efficient design disclosed herein, lightweight and relatively inexpensive materials can be utilized. For example, relatively inexpensive, low-friction materials are ideal materials for ratcheting mechanisms incorporating features of the present disclosure. Some ideal materials include Polytetrafluoroethylene (PTFE), often marketed under the brand name Teflon®, nylon polymers, Polyetherimide), often marketed under the brand name Ultem®, Polyetherimide with glass, Polyetherimide with Nylon, Polyetherimide with PTFE, Polyetheretherketone, Polyetheretherketone with glass, Polyetheretherketone with carbon, thermal plastics, or combinations thereof. Use of such materials over the more commonly used metal in medical devices is particularly useful for ratcheting mechanisms for use in disposable single-use handles and devices.

Throughout this description, the discussed embodiment and examples illustrated should be considered as exemplars, rather than as limitations on the present disclosure. As used herein, the term “disclosure,” “device,” “present disclosure,” or “present device” refers to any one of the embodiments of the disclosure described herein, and any equivalents. Furthermore, reference to various feature(s) of the “disclosure,” “device,” “present disclosure,” or “present device” throughout this document does not mean that all claimed embodiments or methods must include the referenced feature(s).

Furthermore, any element in a claim that does not explicitly state “means for” performing a specified function, or “step for” performing a specific function, is not to be interpreted as a “means” or “step” clause as specified in 35 U.S.C. § 112, for example, in 35 U.S.C. § 112(f) or pre-AIA 35 U.S.C. § 112, sixth paragraph. In particular, the use of “step of” or “act of” in the claims herein is not intended to invoke the provisions of 35 U.S.C. § 112.

It is also understood that when an element or feature is referred to as being “on” or “adjacent” to another element or feature, it can be directly on or adjacent the other element or feature or intervening elements or features may also be present. It is also understood that when an element is referred to as being “attached,” “connected” or “coupled” to another element, it can be directly attached, connected or coupled to the other element or intervening elements may be present. In contrast, when an element is referred to as being “directly attached,” “directly connected” or “directly coupled” to another element, there are no intervening elements present.

Please note if used relative terms such as “left,” “right,” “front,” “back,” “top,” “bottom'” “forward,” “reverse,” “clockwise,” “counter-clockwise,” “outer,” “inner,” “above,” “upper,” “lower,” “below,” “horizontal,” “vertical,” and similar terms, have been used for convenience purposes only and are not intended to imply any particular fixed direction. Instead, they are used to reflect relative locations and/or directions between various portions of an object.

Although ordinal terms, e.g., first, second, third, etc., may be used herein to describe various elements or components, these elements or components should not be limited by these terms. These terms are only used to distinguish one element or component from another element or component. Thus, a first element or component discussed below could be termed a second element or component without departing from the teachings of the present disclosure.

The terminology used herein is for describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” when used herein, specify the presence of stated features, integers, steps, operations, elements, and/or components, but do not preclude the presence or addition of one or more other features, integers, steps, operations, elements, components, and/or groups thereof.

Embodiments of the disclosure are described herein with reference to different views and illustrations that are schematic illustrations of idealized embodiments of the disclosure. As such, variations from the shapes of the illustrations as a result, for example, of manufacturing techniques and/or tolerances are expected. Embodiments of the disclosure should not be construed as limited to the particular shapes of the regions illustrated herein, but are to include deviations in shapes that result, for example, from manufacturing.

It is understood that when a first element is referred to as being “between” or “interposed between” two or more other elements, the first element can be directly between the two or more other elements or intervening elements may also be present between the two or more other elements. For example, if a first element is “between” or “interposed between” a second and third element, the first element can be directly between the second and third elements with no intervening elements, or the first element can be adjacent to one or more additional elements with the first element and these additional elements all between the second and third elements.

FIG. 1 shows an embodiment of a ratcheting medical screwdriver device 100, incorporating features of the present disclosure. The ratcheting medical screwdriver device 100 comprises a housing or body 102 having a proximal end 104 and a distal end 106. The housing 102 comprises a handle portion 108 at the proximal end 104 and comprises an operational component 110 at the distal end 106. The operational component 110 is configured to engage with a particular medical implant, for example, an orthopedic bone screw. The handle portion 108 allows a user, such as a surgeon, to grip the handle portion 108 and apply torque and force to it, which can in turn transmit the torque and force to the operational component 110, which can then apply the torque and force to a medical implant, for example, to tighten an orthopedic bone screw.

Between the operational component 110 and the handle portion 108, the ratcheting medical screwdriver 100 comprises a ratcheting component 112 (also interchangeably referred to herein as the “ratcheting mechanism”), which is configured to allow ratcheting of the medical screwdriver 100. For example, the ratcheting component 112 can be configured such that rotational movement of the handle portion 108 by a user in the clockwise direction will transmit force and torque to the operational component 110, causing similar clockwise movement, for example, allowing for tightening of a medical implant in the clockwise direction.

In some embodiments, the ratcheting component 112 can be configured to only allow the transfer of force and torque in the clockwise direction and will disallow transfer of force and torque in the counterclockwise direction. This can allow a user to rotate the handle 108, and therefore his grip on the handle, counterclockwise, without causing a transfer of the counterclockwise movement to the operational component 110, which could cause a loosening of the engaged medical implant.

In other embodiments the ratcheting component 112 can be configured in an inverse manner, that is, to only allow the transfer of force and torque in the counterclockwise direction and will disallow transfer of force and torque in the clockwise direction. In some embodiments the ratcheting component 112 can be configured to be toggled between two modes, a first mode allowing only clockwise transmission of force and torque and a second mode allowing only counterclockwise transmission of torque and force.

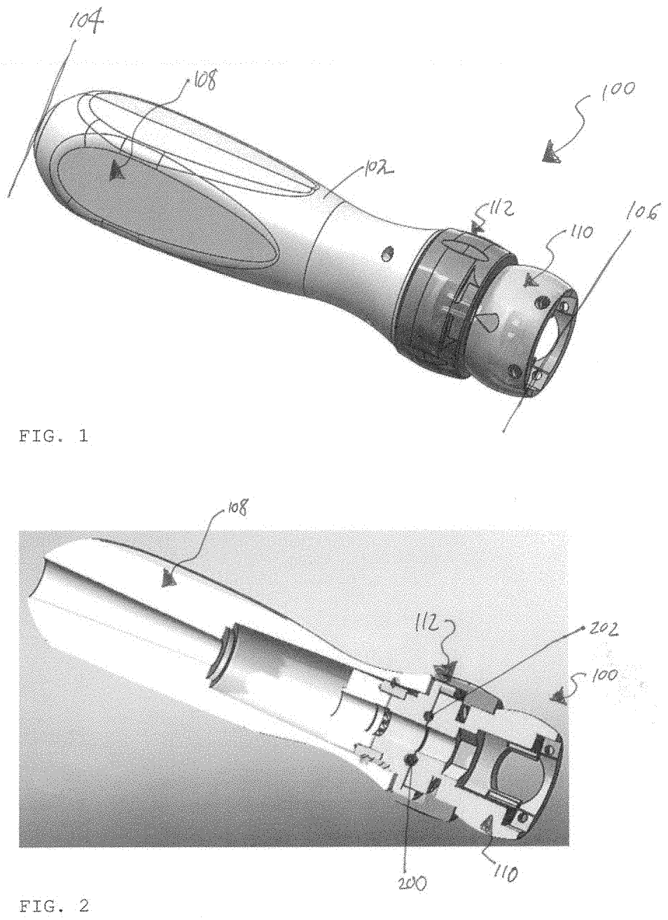

FIG. 2 shows a sectional view of the embodiment of the ratcheting medical screwdriver 100 disclosed in FIG. 1, comprising the body 102, the handle portion 108, the operational component 110 and the ratcheting component 112. In this sectional view, one or more optional ball bearings 200, within an optional race 202 are shown. The ratcheting component 112 can comprise the ball bearings 200 within the race 202 that can increase axial stability and reduce or eliminate collar friction caused by normal operational use of the ratcheting medical screwdriver 100.

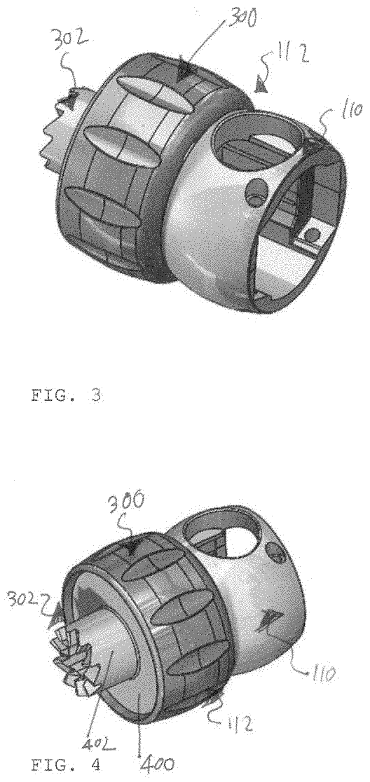

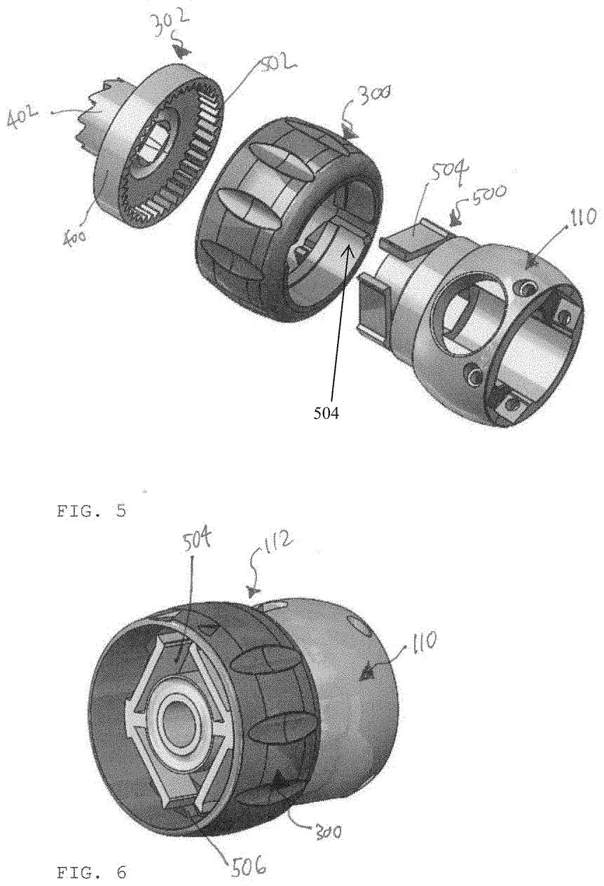

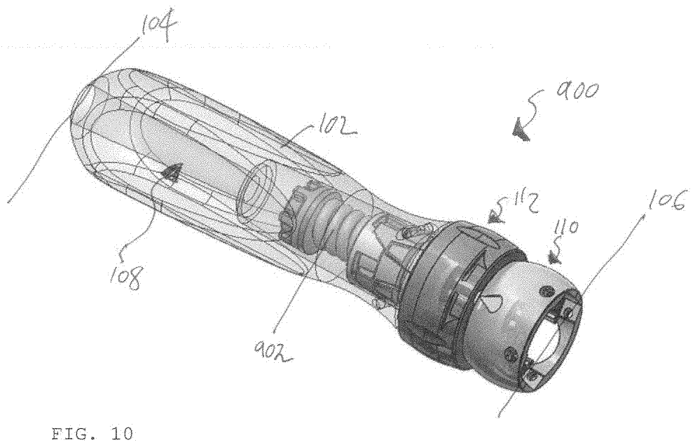

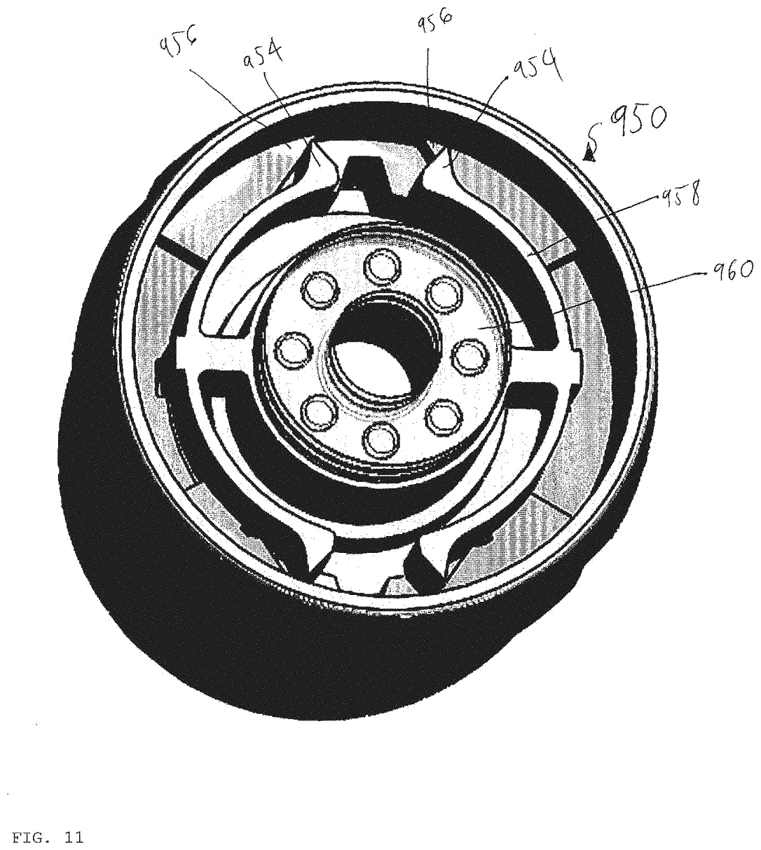

FIG. 3 shows a more detailed view of the outer components of the embodiment of the ratcheting component 112 and the operational component 110 disclosed in FIG. 1. FIG. 3 shows the operational component 110, being connected to a toggle collar 300 and a moveable component 302, which can connect to the handle portion 108 shown in FIG. 1-2 above. FIG. 4 shows a spatially rotated version of the ratcheting mechanism of FIG. 3, and shows that the moveable component 302 can comprise a moveable component body 400 and an optional adapter portion 402, which can connect the moveable portion to the handle portion 108 (shown in FIG. 1-2) in embodiments wherein the handle portion 108 is not connected to the moveable component body 400 via another structure, or can connect the moveable component to additional optional features such as, for example, a torque limiter, as shown in FIG. 10.

FIG. 5 shows an exploded view of the distal end 106 of one embodiment of the ratcheting medical screwdriver device, and, more specifically, an exploded view of the ratcheting component 112, comprising: the moveable component 302, an engagement component 500, and a toggle collar 300 in between the moveable component 302 and the engagement component 500. FIG. 5 shows the moveable component 302 comprising a plurality of teeth 502, as well as the moveable component body 400, and the optional adapter portion 402. The engagement component 500 comprises a plurality of protrusions 504, which can be any protruding structure configured to engage with the plurality of teeth 502 of the moveable component 302. In the embodiment shown in FIG. 5, the protrusions 504 comprise leaf springs. In some embodiments the protrusions are identical or near identical structures, like the embodiment shown, wherein each protrusion is a leaf spring. In other embodiments, the plurality of protrusions can comprise different structures, for example, some protrusions can be leaf springs, while others can be simple protruding structures.

FIG. 5 further shows that in some embodiments the toggle collar 300 can comprise one or more stops 506, configured to engage the protrusions 504 of the engagement component 500. The operation of this engagement between the stops 506 and the protrusions 504 is better illustrated and described with regard FIGS. 8 and 9 below.

As shown in FIG. 5, in some embodiments, the engagement component 500 can be connected to the operational component 110, for example via an intermediate structure or, as shown in FIG. 5, it can be formed integral to the operational component 110.

FIG. 6 is a rear perspective view of the ratcheting component 112 showing the operational component 110, the toggle collar 300, which can at least partially surround the protrusions 504 of the engagement component 500. Also shown are the stops 506, which can be formed integrally to the toggle collar 300, such that rotation of the toggle collar 300 causes a change in position of the stops 506, and therefore changes the mode by which the stops 506 engage with the protrusions 504.

This in turn changes how the teeth 502 interact with the protrusions 504, for example, dictating whether the teeth 502 can slip past the protrusions 504 without catching and thereby rotate the moveable component 302 without transfer of rotational motion and force to the engagement component 500 and the connected operational component 110 or whether the teeth can catch on the protrusions 504 and therefore impart rotational force and motion to the engagement component 500 and the connected operational component 110. This allows for modes of ratcheting wherein rotating the toggle collar 300 in a first direction can initiate a first operational mode wherein clockwise rotation of the movement component 302 is transferred, but counterclockwise rotational movement is not, and rotation of the toggle collar in a second direction can initiate a second operational mode wherein clockwise rotational movement of the movement component 302 is not transferred, but counterclockwise rotational movement is transferred.

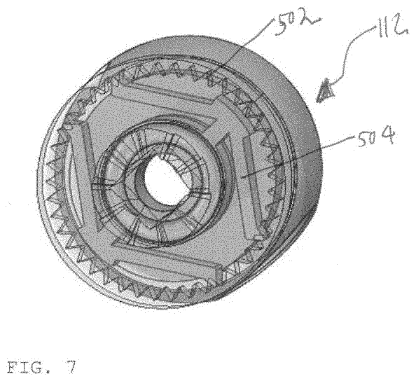

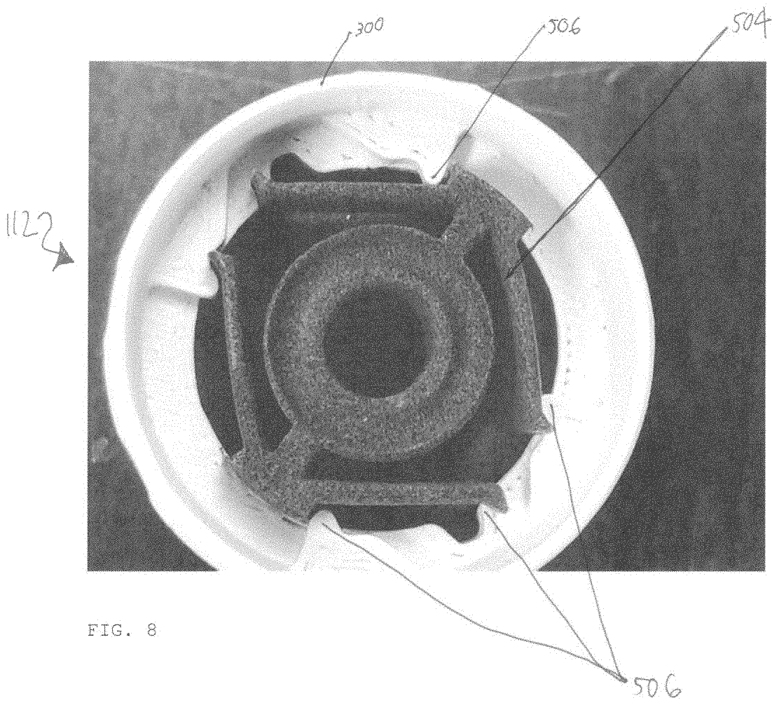

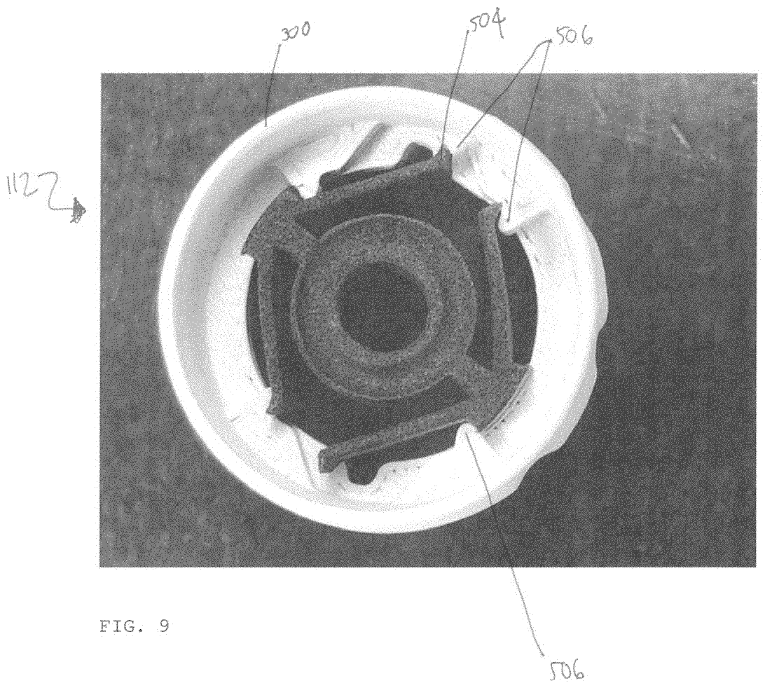

FIG. 7 shows a rear schematic view of the ratcheting component 112, which helps illustrate how the teeth 502 of the moveable component can come into contact to engage the protrusions 504 of the engagement component. The operational nature of the toggle collar 300 is shown in more detail in FIGS. 8 and 9, which show a rear view of the ratcheting component 112, with the movement portion 302 removed to better view the action of the toggle collar 300. In the embodiment of FIG. 8, the toggle collar 300 has been rotated such that connected stops 506 (integrally formed in the embodiment shown) engage with the protrusions 504 of the engagement component in such a manner that clockwise motion of a connected movement component will be transferred due to the positioning of the protrusions 504 against the stops 506, such that the teeth of a connected moveable component will lock against the protrusions 504 in the clockwise direction an push against them without slipping, thereby imparting clockwise rotational force and motion. In this configuration counterclockwise motion will not be transferred. As the stops 506 do not position the abutting protrusions 504 to transfer motion in that direction.

In contrast, FIG. 9 shows the toggle collar 300 has been rotated such that connected stops 506 (integrally formed in the embodiment shown) engage with the protrusions 504 of the engagement component in such a manner that counterclockwise motion of a connected movement component will be transferred due to the positioning of the protrusions 504 against the stops 506, such that the teeth of a connected moveable component will lock against the protrusions 504 in the counterclockwise direction and push against them without slipping, thereby imparting counterclockwise rotational force and motion. In this configuration clockwise motion will not be transferred. As the stops 506 do not position the abutting protrusions 504 to transfer motion in that direction.

FIG. 10 shows another embodiment of a ratcheting medical screwdriver 900, similar to ratcheting medical screwdriver 100 in FIG. 1 above, wherein like features are denoted by like reference numbers. The ratcheting medical screwdriver 900 comprises a housing 102 having a proximal end 104 and a distal end 106, and comprises a handle portion 108, an operational component 110 and a ratcheting component 112 there between. In this particular embodiment, the medical screwdriver 900 includes an optional torque-limiter 902, which can be connected to the moveable component of the ratcheting mechanism 112. This can create a ratcheting scheme whereby tightening (e.g., when motion is being transferred) encompasses torque release limits. Examples of such torque limiting devices can be found in, for example, U.S. Pat. No. 10,730,171 and other patents and applications assigned to ECA Medical Instruments, the assignee of the present application.

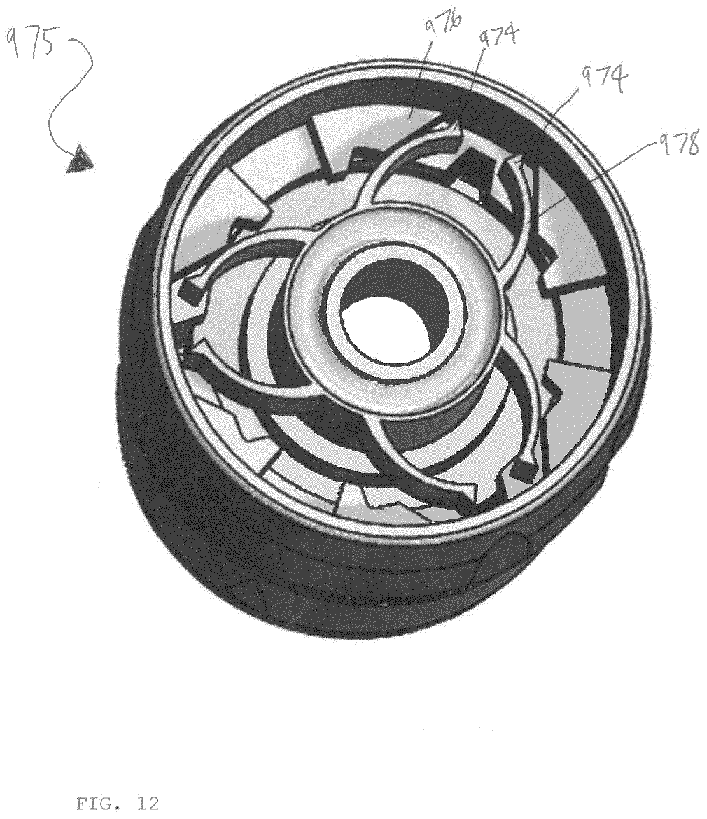

FIG. 11 shows another embodiment of a ratcheting component 950 for integration into a ratcheting medical screwdriver, wherein the ratcheting component 950 is similar to the ratcheting component 112 discussed with regard to the FIGS. 1-10 herein, wherein like reference numbers denote like features. The ratcheting component 950 in FIG. 11 comprises one or more protrusions 954 and one or more stops 956, which function similarly to the protrusions 504 and the stops 506 (best shown in FIGS. 8 and 9) discussed above. The ratcheting component 950 of FIG. 11 differs from the ratcheting component 112 in that, for example, the ratcheting component 950 comprises protrusions 954 that have curved arm portions 958. In this particular embodiment, the curved arm portions 958 are substantially concentric with the body of the ratcheting component 950. This differs from the arm portions of the protrusions 504 of ratcheting component 112, which are substantially straight as shown in FIGS. 8 and 9. It is understood that in embodiments of ratcheting component incorporating features of the present disclosure, the protrusions can comprise any suitable shape that can interact with corresponding stops to provide the desired function. One exemplary advantage of the curved arm portions 958 is that they can provide more flexibility in the protrusions for reduced ratchet force and reverse torque. Furthermore, the curved arm portion 958 effectively reduces the stiffness of the arm portion 958 because the length has a cubed relationship with stiffness.

Another optional feature included in FIG. 11 is the thrust bearing 960, which can provide advantages similar to those of the ball bearing and race configuration shown in FIG. 2 and discussed herein. One advantage of utilizing the thrust bearing 960 rather than a ball bearing and race configuration is that it may be easier to assemble the ratcheting component 950.

FIG. 12 shows another embodiment of a ratcheting component 975 for integration into a ratcheting medical screwdriver, wherein the ratcheting component 975 is similar to the ratcheting component 950 discussed with regard to the FIG. 11 herein, wherein like reference numbers denote like features. The ratcheting component 975 in FIG. 12 comprises one or more protrusions 974 and one or more stops 976, which function similarly to the protrusions 954 and the stops 956 discussed above with regard to FIG. 11. The ratcheting component 975 of FIG. 12 differs from the ratcheting component 950 in FIG. 11 in that, for example, the ratcheting component 975 comprises protrusions 974 that have split curved arm portions 978 that are facing each other in an opposing manner as shown.

It is further understood that ratcheting mechanisms incorporating features of the present disclosure can be integrated into various screwdriver devices, for example, devices comprising handles and body portions comprising a variety of shapes and dimensions, for example shapes corresponding to any regular or irregular polygon. Furthermore, it is understood, that devices incorporating features of the present disclosure can incorporate modular features, for example, a screwdriver incorporating features of the present disclosure can comprise a base body, which can further comprise an embodiment of a disclosed ratcheting mechanism. This base body can comprise an operational component that can be removed/disconnected and replaced with an operational component designed to engage with a different type of fastener. Likewise, the base body can comprise a removable/detachable handle that can be exchanged for a different handle better suited for a particular task at hand. One use for this is in embodiments wherein single-use disposable handles might be desired.

It is understood that while the present application is primarily directed to ratcheting mechanisms utilized with medical instruments, it is understood that ratcheting mechanisms incorporating features of the present disclosure can also be utilized in any other tool that can potentially benefit from them, for example, basic screwdrivers for use with construction projects.

We claim:

1. A ratcheting medical screwdriver, comprising:
a housing comprising a proximal and distal end;
a handle portion at said proximal end, said handle portion configured to transmit motion and torque;
an operational component at said distal end, said operational component configured to engage a medical implant; and
a ratcheting mechanism between said operational component and said handle, said ratcheting mechanism configured to allow torque generated by movement from said handle in a first direction to be transmitted to said operational component and to prevent torque generated by movement from said handle in a second direction to be transmitted to said operational component.

2. The ratcheting medical screwdriver of claim 1, wherein said ratcheting mechanism comprises Polytetrafluoroethylene (PTFE).

3. The ratcheting medical screwdriver of claim 1, wherein said ratcheting mechanism comprises nylon polymer.

4. The ratcheting medical screwdriver of claim 1, wherein said ratcheting mechanism comprises:
a moveable component connected to said handle portion, said moveable component comprising a plurality of teeth;
an engagement component connected to said operational component, said engagement component comprising protrusions configured to engage with said plurality of teeth; and
a toggle collar between said moveable component and said engagement component, said toggle collar comprising a plurality of stops configured to engage with said protrusions.

5. The ratcheting medical screwdriver of claim 4, wherein when said toggle collar is in a first operational position, said stops engage said protrusions such that said protrusions engage with said plurality of teeth to allow torque generated by movement from said handle in said first direction to be transmitted to said operational component and to prevent torque generated by movement from said handle in said second direction to be transmitted to said operational component.

6. The ratcheting medical screwdriver of claim 4, wherein when said toggle collar is in a second operational position, said stops engage said protrusions such that said protrusions engage with said plurality of teeth to prevent torque generated by movement from said handle in said first direction to be transmitted to said operational component and to allow torque generated by movement from said handle in said second direction to be transmitted to said operational component.

7. The ratcheting medical screwdriver of claim 5, wherein said first direction is the clockwise direction and said second direction is the counterclockwise direction.

8. The ratcheting medical screwdriver of claim 4, wherein said protrusions comprise leaf springs.

9. The ratcheting medical screwdriver of claim 4, wherein said protrusions are integral with said operational component.

10. The ratcheting medical screwdriver of claim 4, wherein said plurality of teeth comprise at least 40 teeth.

11. The ratcheting medical screwdriver of claim 1, wherein said ratcheting mechanism further comprises one or more ball bearings configured with a race within said ratcheting mechanism.

12. The ratcheting medical screwdriver of claim 4, wherein said toggle collar substantially surrounds said protrusions and said teeth.

13. The ratcheting medical screwdriver of claim 4, further comprising a torque-limiter, wherein said moveable component is connected to said torque-limiter.

14. A ratcheting mechanism for use in a medical screwdriver, comprising:
a moveable component configured to connect to a handle portion of a medical screwdriver, said movement component comprising a plurality of teeth;
an engagement component configured to connect to an operational component of said medical screwdriver, wherein said operational component is configured to engage a medical implant, said engagement component comprising protrusions configured to engage with said plurality of teeth; and
a toggle collar between said moveable component and said engagement component, said toggle collar comprising a plurality of stops configured to engage with said protrusions.

15. The ratcheting mechanism of claim 14, wherein said protrusions comprise leaf springs.

16. The ratcheting mechanism of claim 14, wherein said toggle collar substantially surrounds said protrusions and said teeth.

17. The ratcheting mechanism of claim 14, wherein said ratcheting mechanism comprises Polytetrafluoroethylene (PTFE).

18. A ratcheting medical screwdriver, comprising:
a housing comprising a proximal and distal end;
a handle portion at said proximal end, said handle portion configured to transmit motion and torque;
an operational component at said distal end, said operational component configured to engage a medical implant;
a ratcheting mechanism between said operational component and said handle, said ratcheting mechanism comprising:
a moveable component connected to said handle portion, said moveable component comprising a plurality of teeth;
an engagement component connected to said operational component, said engagement component comprising protrusions configured to engage with said plurality of teeth; and
a toggle collar between said moveable component and said engagement component, said toggle collar comprising a plurality of stops configured to engage with said protrusions.

19. The ratcheting medical screwdriver of claim 18, wherein said ratcheting mechanism comprises Polytetrafluoroethylene (PTFE).

20. The ratcheting medical screwdriver of claim 19, wherein said protrusions comprise leaf springs.
